(19) United States
(12) Patent Application Publication
(10) Pub. No.: US 2024/0369569 A1
(43) Pub. Date: Nov. 7, 2024

(54) Prediction of Severity of COVID-19 and Methods of Treatment

(71) Applicant: Vanda Pharmaceuticals Inc., Washington, DC (US)

(72) Inventors: Sandra Smieszek, Washington, DC (US); Vasilios Polymeropoulos, Washington, DC (US); Christos Polymeropoulos, Washington, DC (US); Bartlomiej Przychodzen, Washington, DC (US); Mihael Polymeropoulos, Washington, DC (US)

(21) Appl. No.: 18/714,763

(22) PCT Filed: Dec. 2, 2022
(86) PCT No.: PCT/US2022/080795

§ 371 Date: May 30, 2024

Related U.S. Application Data
(60) Provisional application No. 63/264,896, filed on Dec. 3, 2021.

(51) Int. Cl. G01N 33/68

(52) U.S. Cl. CPC G01N 33/6863; G01N 2333/521; G01N 2800/52

(57) ABSTRACT

The invention relates generally to coronavirus disease 2019 (COVID-19) and more particularly to the prediction of severity of COVID-19 manifestation in a patient or in an individual at risk of infection by severe acute respiratory syndrome coronavirus 2 (SARS-COV-2) based on a level of chemokine (C-X-C motif) ligand 16 (CXCL 16) expression in and/or the rs 10490770 genotype of the patient or individual.

### CROSS-REFERENCE TO RELATED APPLICATIONS

This application claims priority to co-pending U.S. Provisional Patent Application Ser. No. 63/264,896, filed 3 Dec. 2021, the entirety of which is incorporated herein as though fully set forth.

### BACKGROUND

### SARS-COV-2

Severe acute respiratory syndrome coronavirus 2 (SARS-COV-2 or sometimes 2019-nCOV), a spherical positive single-stranded RNA virus, has caused an ongoing pandemic of coronavirus disease 2019 (COVID-19). To date, SARS-COV-2 has infected over 126 million individuals worldwide and resulted in over 2.7 million deaths. SARS-COV-2 is a strain of the SARS-COV species in the betacoronavirus genus.

As with other coronaviruses, SARS-COV-2 has four structural proteins—the S (spike), E (envelope), M (membrane), and N (nucleocapsid). The S, E, and M proteins form the viral envelope, while the N protein, located in the core of the viral particle, binds the viral RNA and is responsible for its conformation into the viral particle. Some betacoronaviruses also include a hemagglutinin-esterase (HE) protein on the particle surface, which may enhance entry into the host cell.

The spike proteins are trimeric class I fusion proteins, heavily glycosylated, and project from the virion surface, promoting attachment to and entry into host cells. In some coronaviruses, the S protein consists of two subunits, S1 and S2, on the surface of the viral particle. In other coronaviruses, including SARS-COV-2, the S protein includes S1 and S2 domains but remains intact on the viral particle surface until cleavage inside endocytic vesicles during viral entry.

Significant structural rearrangement of the S protein is involved in its fusion with a host cell membrane. Specifically, the receptor-binding domain (RBD) of the S1 subunit of the S protein undergoes a conformational movement from a “down” conformation, in which the binding receptors of the S1 subunit are inaccessible, to an “up” conformation, in which the receptors are accessible. The “up” conformation is believed to be less stable than the “down” conformation.

The S2 domain controls entry of the virus into the host cell. Angiotensin-converting enzyme 2 (ACE2), a type I membrane protein, is expressed widely across human tissues, including lung, heart, kidney, intestine, and adipose tissue. ACE2 has been identified as the host cell receptor for the earlier SARS-COV strain and is a target of the SARS-COV-2 S protein RBD. RBD binding is believed to occur on the outer surfaces of the ACE2 protein, while angiotensin substrate binding occurs within a deep cleft of the protein.

Recently, the 3.5-angstrom-resolution structure of the S protein has been described. As noted above, the S protein is cleaved into two its S1 and S2 subunits. This cleavage of S proteins by host proteases is critical for viral infection and its exit from the cell via lysosomes.

During infection, the S protein is cleaved by host cell proteases, exposing a fusion peptide of the S2 domain. Cleavage of the S protein occurs between the S1 and S2 domains and subsequently within the S2 domain (S2′) proximal to the fusion peptide. This leads to the fusion of viral and cellular membranes and the release of the viral genome into the cytoplasm of the host cell. Cleavage at both sites is believed to be necessary for viral entry into a host cell.

### CXCR6 and CXCL16

Recent genome-wide association studies have identified a gene cluster on chromosome 3 (3p21.31; rs73064425), which includes the C-X-C motif chemokine receptor 6 (CXCR6) gene, with an increased risk for severe manifestations of COVID-19. This DNA segment is approximately 50 kb in size and includes, in addition to the CXCR6 gene, LZTFL1, SLC6A20, FYCO1, CCR9, and XCR1. Another variant, rs10490770 (T>C) has been associated with higher rates of hospitalization, critical illness (concluding sever respiratory failure and venous thromboembolism), and death. Genetic variations within this segment and associated with severe COVID were inherited from Neanderthals and are carried by a high proportion of individuals in South Asia and Europe.

Chemokine (C-X-C motif) ligand 16 (CXCL16) is synthesized as a transmembrane molecule and expressed as a cell surface-bound molecule and as a soluble chemokine. CXCL16 interacts with CXCR6 in leukocytes and other cells promoting chemotaxis or cell adhesion. Inflammatory cytokines such as IFNγ and TNFα promote CXCL16 expression. CXCL16 has previously been implicated in the pathogenesis of lung injury, upon which it is released and functions as a chemoattractant for CXCR6+T, natural killer (NK), B, and dendritic cells. CXCL16 levels have been shown to be elevated in the serum of acute lung injury patients.

### SUMMARY

In one embodiment, the invention provides a method of treating a patient diagnosed with coronavirus disease 2019 (COVID-19), the method comprising: determining a level of expression of chemokine (C-X-C motif) ligand 16 (CXCL16) in the patient; and in the case that the level of CXCL16 expression is associated with a more severe manifestation of COVID-19, treating the patient for the more severe manifestation of COVID-19, regardless of the patient's actual severity of manifestation of COVID-19.

In another embodiment, the invention provides a method of treating a patient diagnosed with coronavirus disease 2019 (COVID-19), the method comprising: determining that the patient has a genotype at the rs 10490770 single nucleotide polymorphism (SNP) locus associated with a more severe manifestation of COVID-19; and treating the patient for the more severe manifestation of COVID-19, regardless of the patient's actual severity of manifestation of COVID-19.

In still another embodiment, the invention provides a method of predicting a severity of manifestation of coronavirus disease 2019 (COVID-19) in a patient diagnosed with COVID-19 or at risk of infection by severe acute respiratory syndrome coronavirus 2 (SARS-COV-2), the method comprising: determining a level of expression of chemokine (C-X-C motif) ligand 16 (CXCL16) in the patient; and in the case that the level of CXCL16 expression is associated with a more severe manifestation of COVID-19, predicting that the patient will experience a more severe manifestation of COVID-19; or in the case that the level of CXCL16 expression is not associated with a more severe manifestation of COVID-19, predicting that the patient will experience a less severe manifestation of COVID-19.

In still yet another embodiment, the invention provides a method of predicting a severity of manifestation of coronavirus disease 2019 (COVID-19) in a patient diagnosed with COVID-19 or in an individual at risk of infection by severe acute respiratory syndrome coronavirus 2 (SARS-COV-2), the method comprising: determining the patient's or individual's rs 10490770 genotype; and in the case that the patient's or individual's rs10490770 genotype is determined to be TT, predicting that the patient or individual is likely not to experience a severe manifestation of COVID-19; or in the case that the patient's or individual's rs10490770 genotype is determined to be non-TT, predicting that the patient or individual is likely to experience a severe manifestation of COVID-19.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which: FIG. 1 shows baseline plasma CXCL16 levels among COVID-19 patients and COVID-negative control subjects;

FIG. 2 shows baseline plasma CXCL16 levels among COVID-19 patients who died and survived the disease;

FIG. 3 shows baseline plasma CXCL16 levels among COVID-19 patients according to severity of manifestation of COVID-19; and

FIG. 4 shows a plot of CXCL16 concentration, BMI, and age in COVID-19 patients who survived and died, showing a correlation with CXCL16 concentration.

It is noted that the drawings of the invention are not to scale. The drawings are intended to depict only typical aspects of the invention, and therefore should not be considered as limiting the scope of the invention. In the drawings, like numbering represents like elements among the drawings.

### DETAILED DESCRIPTION

Applicant's ODYSSEY study is a double-blinded Phase 3 study with a planned randomization of a total of 300 hospitalized severely ill COVID-19 patients. Inclusion criteria for the study include:
1. Adults aged 18-90;
2. confirmed laboratory COVID-19 infection;
3. confirmed pneumonia by chest radiograph or computed tomography;
4. fever defined as temperature≥36.6° C. armpit, ≥37.2 C oral, or ≥37.8° C. rectal since admission or the use of antipyretics;
5. PaO₂/FiO₂≤300; and
6. In patient hospitalization.
   Main Exclusion Criteria include:
1. Inability to provide informed consent or to have an authorized relative or designated person provide informed consent, or to comply with the protocol requirements;
2. Known allergy to tradipitant or other neurokinin-1 antagonists;
3. Pregnancy;
4. Uncontrolled HIV, HBV, or HCV infection;
5. Other uncontrolled medically significant diseases;
6. Enrollment in another clinical trial of an investigational therapy;
7. Alanine aminotransferase>5× Upper Limit of Normal or Creatinine clearance<50 m 1741/min;
8. Requiring mechanical ventilation for >72 hours.

Patients are followed for up to 28 days to record clinical outcomes. Clinical progress is recorded on a 7-point clinical status defined as:
1—Death;
2—Hospitalized on mechanical ventilation or ECMO;
3—Hospitalized on non-invasive ventilation or high-flow oxygen supplementation;
4—Hospitalized requiring supplemental oxygen;
5—Hospitalized not requiring supplemental oxygen, requiring continued medical care;
6—Hospitalized not requiring supplemental oxygen, not requiring continued medical care;
7—Not hospitalized.

Details of the COVID-19 patients and COVID-negative control subjects are shown below in Tables 1 and 2.

TABLE 1

| | |
|---|---|
| Average Age (years) | 64 (54-72) |
| Sex, n (%) | |
| Male | 91 (64.5) |
| Female | 50 (35.5) |
| Race, n (%) | |
| Asian | 2 (1.4) |
| Black or African-American | 35 (24.8) |
| Native Hawaiian or Other Pacific Islander | 1 (0.7) |
| White | 73 (51.8) |
| Other | 30 (11.3) |
| Comorbidities, n (%) | 135 (95.7) |
| Hypertension | 80 (56.7) |
| Diabetes | 56 (39.7) |
| Coronary Heart Disease | 10 (7.1) |
| Asthma | 16 (11.3) |
| WHO ordinal scale at baseline, n (%) | |
| 2-hospitalized on mechanical ventilation or ECMO | 8 (5.7) |
| 3-hospitalized on non-invasive ventilation or high- | 69 (48.9) |
| flow oxygen supplement | |
| 4-hospitalized requiring supplemental oxygen | 61 (43.3) |
| 5-hospitalized not requiring supplemental oxygen, | 3 (2.1) |
| requiring continued medical care | |

TABLE 2

| | |
|---|---|
| Average time from hospitalization to starting | 2 (2-7) |
| study treatment, days, n (%) | |
| Early (10 days from hospitalization) | 127 (90.1) |
| Late (>10 days from hospitalization) | 14 (9.9) |
| Highest oxygen therapy support, n (%) | |
| Room air | 3 (2.1) |
| Nasal cannula | 46 (32.6) |
| Non-rebreather | 15 (10.6) |
| High flow nasal cannula | 52 (36.9) |
| CPAP mask | 7 (5.0) |
| BiPAP mask | 10 (7.1) |
| Mechanical ventilation | 8 (5.7) |
| Concomitant medications, n (%) | 39 (27.7) |
| Azithromycin | 19 (13.5) |
| Tocilizumab | 4 (2.8) |
| Hydroxychloriquine | 12 (8.5) |
| Steroids | 21 (14.9) |

As used herein, a more severe manifestation of COVID-19 means a patient hospitalized on mechanical ventilation or ECMO (WHO=2 in the ODYSSEY study) and less severe manifestations of COVID-19 means any other degree of severity other than death (WHO=3, 4, 5, 6, or 7 in the ODYSSEY study). While patients who have died of COVID-19 are clearly those who have experienced a severe manifestation of COVID-19, for purposes of the methods described herein, e.g., predicting the severity of manifestation of COVID-19 in a living patient and/or treating that living patient, consideration of patients who have already died of COVID-19 is not relevant. Plasma concentrations of CXCL16 of the hospitalized COVID-19 patients not otherwise excluded (n=114) and SARS-COV-2-negative control subjects (n=37) are assessed using an ELISA assay to characterize the CXCR6/CXCL16 axis in the pathogenesis of severe COVID-19. DNA samples of patients and controls are collected for subsequent sequencing.

Results show elevated CXCL16 levels in hospitalized COVID-19 patients. As shown in FIG. 1, baseline CXCL16 levels are higher in COVID-19 patients with a severe manifestation (WHO=2) than patients with less severe manifestations (WHO=3, 4, or 5) (p=0.0235), while the difference between all COVID-19 patients (WHO=2, 3, 4, or 5) and COVID-negative control subjects is not significantly different. CXCL16 expression of about 600 pg/mL or greater is associated with more severe manifestations of COVID-19. These results are not correlated with patient age, BMI, or sex.

Therefore, according to an embodiment of the invention, a patient's severity of COVID-19 infection may be predicted by determining the patient's level of expression of CXCL16. In the case that the level of CXCL16 expression is associated with more severe manifestations of COVID-19 (i.e., 600 pg/mL or greater), it can be predicted that the patient will experience a more severe manifestation of COVID-19.

Contrarily, in the case that the level of CXCL16 expression is not associated with more severe manifestations of COVID-19 (i.e., less than 600 pg/mL), it can be predicted that the patient will experience a less severe manifestation of COVID-19. Such prediction may be made for a patient diagnosed with COVID-19 or an individual at risk of infection by SARS-COV-2 and the develop COVID-19.

Similarly, a patient diagnosed with COVID-19 may be treated based on such a determination. For example, upon determining that the level of CXCL16 expression is associated with more severe manifestations of COVID-19, treatment of the patient may include treating the patient for a more severe manifestation of COVID-19 regardless of the level of severity of manifestation of COVID-19 the patient exhibits. Such treatment may include inhibiting CXC16 activity in the patient by, for example, administering to the patient an anti-CXCL16 antibody in an amount sufficient to inhibit CXCL16 activity in the patient. Both monoclonal and polyclonal CXCL16 antibodies are known and available, for example, from Invitrogen.

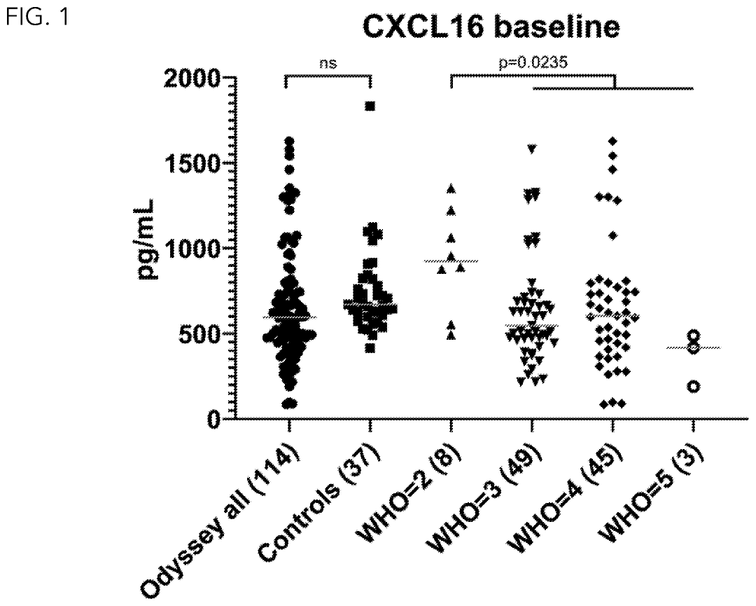

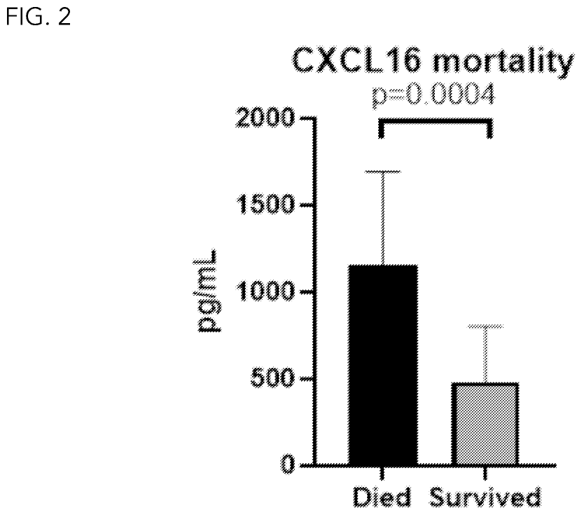

CXCL16 levels are also significantly (p=0.0004) higher among COVID-19 patients who ultimately died of the disease than among those patients who, at the conclusion of the study, had survived. As can be seen in FIG. 2, average CXCL16 levels among patients who ultimately died of COVID-19 are more than twice the average of patients who did not die.

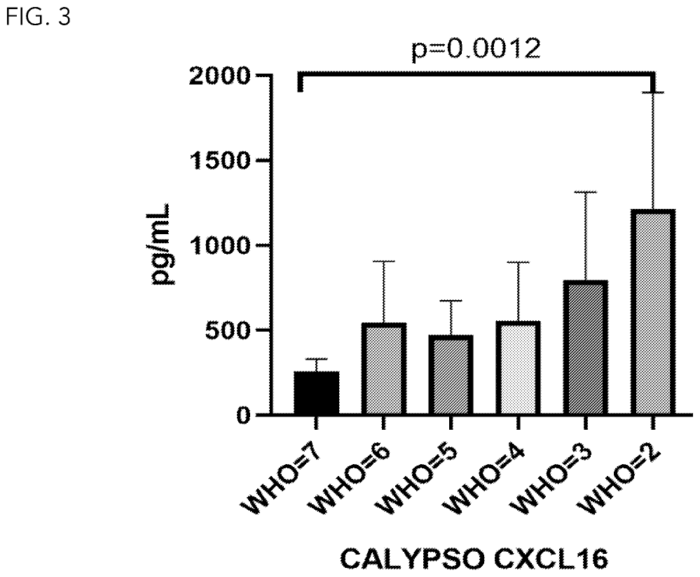

These results are replicated in a subsequent study of 70 COVID-19 patients, as shown in FIG. 3. Here, patients with the most severe manifestation of COVID-19 (WHO=2) again had significantly higher baseline CXCL16 levels than did patients with less severe manifestations (WHO=3-7).

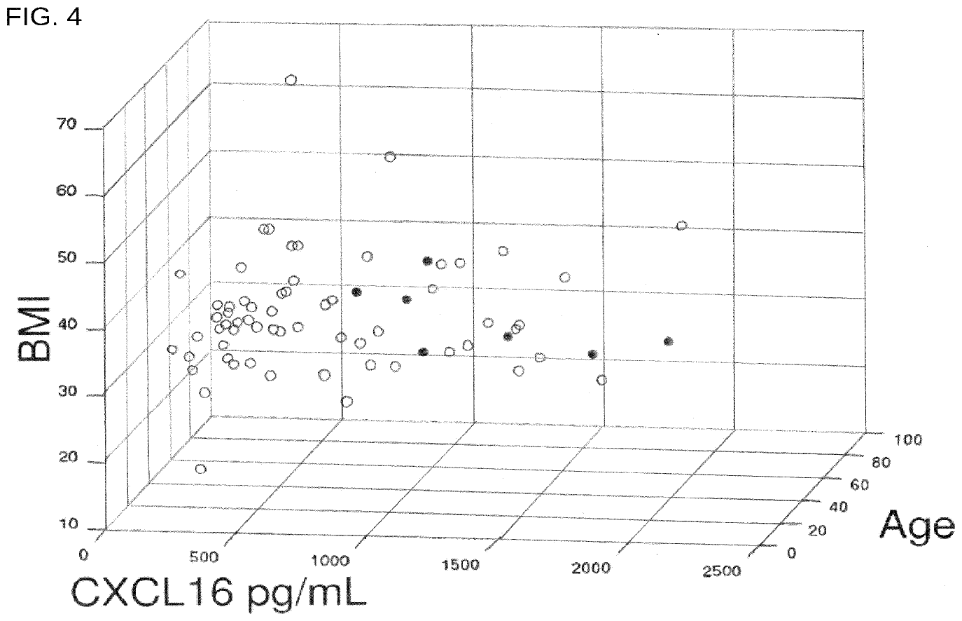

The combined results from both studies suggest that CXCL16 concentration is correlated with COVID-19 mortality. FIG. 4 shows a plot of CXCL16 concentration, BMI, and age of COVID-19 patients. Filled circles represent patients who died of COVID-19.

At a CXCL16 concentration greater than 700 pg/mL, a COVID-19 patient has a mortality rate of approximately 25%. This increases to almost 50% in patients older than 52 with a BMI greater than 26. All patients in the study who died of COVID-19 were in this group.

Results show no correlation between CXCL16 level and IFN-γ or TNF-α expression.

Genetic analysis reveals an association between the rs10490770 variant and severe COVID-19 infection. Carriers of the rs 10490770 T>C variant have a significantly (p<0.002) higher risk of mortality when compared to wildtype patients.

As such, embodiments of the invention may include similar methods of predicting a severity of COVID-19 or treating COVID-19 based upon determination of a patient's rs 10490770 genotype. For example, upon determining that a COVID-19 patient or an individual at risk for SARS-COV-2 infection has an rs 10490770 genotype associated with the more severe manifestation of COVID-19 (i.e., a non-TT genotype), it could be predicted that the patient or individual is likely to experience the more severe manifestation of COVID-19. Contrarily, if the patient or individual is determined to have a TT genotype at the rs 10490770 locus, it could be predicted that the patient or individual is likely to experience a less severe manifestation of COVID-19.

Such prediction may be employed in the treatment of COVID-19 patients. For example, upon determining that the patient has a non-TT rs 10490770 genotype, the patient could be treated for the more severe manifestation of COVID-19, regardless of the patient's actual severity of manifestation of COVID-19. Such treatment may include, for example, inhibiting CXCL16 activity in the patient. Such inhibition may include the administration of an anti-CXCL16 antibody to the patient in an amount sufficient to inhibit CXCL16 activity. As noted above, monoclonal and polyclonal CXCL16 antibodies are known in the art.

As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of treating a patient diagnosed with coronavirus disease 2019 (COVID-19), the method comprising:
determining a level of expression of chemokine (C-X-C motif) ligand 16 (CXCL16) in the patient; and
in the case that the level of CXCL16 expression is associated with a more severe manifestation of COVID-19, treating the patient for the more severe manifestation of COVID-19, regardless of the patient's actual severity of manifestation of COVID-19.

2. The method of claim 1, wherein the level of CXCL16 expression associated with the more severe manifestation of COVID-19 is 600 pg/mL or more.

3. The method of claim 1, wherein treating the patient includes inhibiting CXCL16 activity in the patient.

4. The method of claim 3, wherein inhibiting CXCL16 activity in the patient includes administering to the patient an anti-CXCL16 antibody in an amount sufficient to inhibit CXCL16 activity.

5. A method of treating a patient diagnosed with coronavirus disease 2019 (COVID-19), the method comprising:
determining that the patient has a genotype at the rs 10490770 single nucleotide polymorphism (SNP) locus associated with a more severe manifestation of COVID-19; and
treating the patient for the more severe manifestation of COVID-19, regardless of the patient's actual severity of manifestation of COVID-19.

6. The method of claim 5, wherein the genotype at the rs 10490770 SNP associated with the more severe manifestation of COVID-19 is non-TT.

7. The method of claim 5, wherein treating the patient includes inhibiting CXCL16 activity in the patient.

8. The method of claim 7, wherein inhibiting CXCL16 activity in the patient includes administering to the patient an anti-CXCL16 antibody in an amount sufficient to inhibit CXCL16 activity.

9. A method of predicting a severity of manifestation of coronavirus disease 2019 (COVID-19) in a patient diagnosed with COVID-19 or at risk of infection by severe acute respiratory syndrome coronavirus 2 (SARS-COV-2), the method comprising:
determining a level of expression of chemokine (C-X-C motif) ligand 16 (CXCL16) in the patient; and
in the case that the level of CXCL16 expression is associated with a more severe manifestation of COVID-19, predicting that the patient will experience a more severe manifestation of COVID-19; or
in the case that the level of CXCL16 expression is not associated with a more severe manifestation of COVID-19, predicting that the patient will experience a less severe manifestation of COVID-19.

10. The method of claim 9, wherein the level of CXCL16 expression associated with the more severe manifestation of COVID-19 is 600 pg/mL or more and the level of CXCL16 expression associated with the less severe manifestation of COVID-19 is less than 600 pg/mL.

11. The method of claim 9, further comprising:
in the case that the patient is diagnosed with COVID-19, inhibiting CXCL16 activity in the patient.

12. The method of claim 11, wherein inhibiting CXCL16 activity in the patient includes administering to the patient an anti-CXCL16 antibody in an amount sufficient to inhibit CXCL16 activity.

13. A method of predicting a severity of manifestation of coronavirus disease 2019 (COVID-19) in a patient diagnosed with COVID-19 or in an individual at risk of infection by severe acute respiratory syndrome coronavirus 2 (SARS-COV-2), the method comprising:
determining the patient's or individual's rs10490770 genotype; and
in the case that the patient's or individual's rs 10490770 genotype is determined to be TT, predicting that the patient or individual is likely not to experience a severe manifestation of COVID-19; or
in the case that the patient's or individual's rs10490770 genotype is determined to be non-TT, predicting that the patient or individual is likely to experience a severe manifestation of COVID-19.

14. The method of claim 13, further comprising:
in the case that the patient is diagnosed with COVID-19, treating the patient for the more severe manifestation of COVID-19, regardless of the patient's actual severity of manifestation of COVID-19.

15. The method of claim 14, wherein treating the patient includes inhibiting CXCL16 activity in the patient.

16. The method of claim 14, wherein inhibiting CXCL16 activity in the patient includes administering to the patient an anti-CXCL16 antibody in an amount sufficient to inhibit CXCL16 activity.
